(19) United States
(12) Patent Application Publication
(10) Pub. No.: US 2009/0099501 A1
(43) Pub. Date: Apr. 16, 2009

(54) Spinal Cord's Epidural Space Detection By Using Fiber Optic Technology

(75) Inventors: Yin Chang, Taipei City (TW); Chien-Kun Ting, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 12/113,691

(22) Filed: May 1, 2008

(30) Foreign Application Priority Data
Oct. 15, 2007 (TW) 096138569

(51) Int. Cl. A61N 1/30; A61M 31/00

(52) U.S. Cl. 604/21; 604/19; 604/67

(57) ABSTRACT

The present invention provides a device and a method for effectively processing epidural anesthesia by providing the needle-localization information in real time. The idea of this invention is derived from the concept that different tissues due to their different composition have different optical properties, such as absorbance and reflection, so we can discriminate the tissue types where the needle reaches by their distinctive optical properties. In this device, the inner solid needle of the puncture needle used before is replaced by a hollow needle which filled with optical fibers, and the distal end of those optical fibers are connected with two light sources of different wavelengths and a light analyzer. Thus, while qualifying and comparing the optical properties of those tissues, these device and method can be very helpful during epidural anesthesia and also reduce the risk of the surgery.

### FIELD OF THE INVENTION

The present invention is related to the field of hypodermic needles and more specifically to hypodermic needles that are used to insert catheters or medication into the epidural space.

### DESCRIPTION OF RELATED ART

Epidural anesthesia is a technique to reach the state of anesthetization by injecting some sedative into the epidural space. Those sedative blocks the spinal neural transmission temperately, so this surgery is also called “epidural block”, and is often applied on surgical operations. For anesthesiologists, the most important issue in epidural anesthesia is whether the needle tip is accurately inserted into the correct position (epidural space). As shown in FIG. 1, epidural space is a small lumen located between dural matter and ligamentum flavum that is hard to position and detect precisely, and its' lumen size varies with age and body shape. Normally, the epidural space in the adult lumbar spine is 3-6 mm deep, but in thorax it is only 1˜5 mm, which means it is comparatively easy to cross it and accidentally puncture the dura with the needle. This may in turn cause the post dural puncture headache (PDPH) or severe spinal injury.

At present, anesthesiologists identify the correct position according to the experience and subjective judgment. Traditionally, they identify epidural space by feeling the resistance of ligamentum flavum, but the resistance of ligamentum flavum varies with age, gender and body shape. For example, the tenacity of ligamentum flavum in old man is weak, and those in pregnant women and obese people also have weak resistance. By the way, the personal feeling toward the anesthesiologist also affects this surgery.

As above mentions, there are still lots of unsolved problems in performing epidural anesthesia, thus we here provide a new device and method to improve the objectivity and precision when doing tissue puncturing surgery such as epidural anesthesia, wish to reduce the risk of surgery.

### SUMMARY OF THE INVENTION

The present invention provides a syringe device for tissue puncturing and drug delivery which provides the user with information about the type of tissue at the distal tip to locate the needle in real time. This device comprises: (a) a hypodermic needle having an outer sheath that has a sharp distal tip for puncturing tissue, wherein said outer sheath defines a hollow inner bore; (b) an inner removable optical fiber within said hollow inner bore such that the light can be transmitted through the fiber into the needle and onto the contacted tissue to stimulate a reflective light by the tissue, and also the reflective light can be transmitted back through the needle and the optical fiber; (c) an catheter suitable for insertion into the needle to replace the optical fiber and for fluid delivery; (d) a light source coupled to the optical fiber for production incident light that transmitted in the fiber; and (e) a light analyzer coupled to the optical fiber for receiving and detecting the reflective light emission from the tissue.

The optical fiber of this device is used for transmitting light from the light source to the target tissue and receiving reflective light from that tissue transmitting to the light analyzer, so the fiber can be a single mode fiber, multi mode fibers, or a branch fiber. In a single mode fiber, the fiber transmits incident light and reflective light simultaneously; but in multi mode fibers some fibers are used for incident light transmission and the others for reflective light separately.

Different tissues have different optical properties due to their different composition, such as absorbance and reflectivity, thus we can discriminate the tissue types where the needle reaches by analyzing their distinctive optical properties. Thus, the light analyzer of the present invention analyses the ratio of one reflective light to another, wherein the two reflective lights are produced by two incident lights on different wavelengths targeting to the same tissue, to provide the tissue type information. More specificity, the analyzer analyses the absorbency of the target tissue between two different lights. In one embodiment, if someone wishes to apply this device on epidural anesthesia, to the inventors' practical experience, the better light wavelengths for discrimination are one light wavelength from 620 nm to 680 nm and the other from 520 nm to 600 nm. In the best embodiment, one wavelength is 650 nm and the other is 580 nm. But in practical, there are some commercial products can emit 650 nm and 532 nm laser, so using these two wavelength light sources are more convenient. Besides, this light analyzer can also notify the user by sound or light when the distal tip of needle reaches the target tissue.

The optical fiber of this device is pulled out from the hypodermic needle and is replaced by the catheter for fluid delivery when the needle reaches a target tissue, and after the catheter been placed the hypodermic needle also been drew out just as the traditional epidural anesthesia. The hypodermic needle and the catheter of this device are both suitable for clinical use.

The light source of this device is a laser source, which emits at least two lights at different wavelengths.

The present invention also provides a method to position a hypodermic needle in real time through optical technology. This method comprises: (a) inserting a removable optical fiber into a hollow hypodermic needle which has a sharp distal tip for puncturing tissue; (b) connecting the optical fiber with a light source and a light analyzer, wherein the optical fiber is capable of transmitting the incident light to the tissue and collecting the reflective light to the light analyzer; (c) transmitting at least two different incident lights to the tissue; and (d) using the light analyzer to analyze the light absorbency of the tissue. By the way, the above said device can also be applied to this method to achieve the goal for effectively processing epidural anesthesia.

When this method is applied to epidural anesthesia, the inner optical fiber of the needle is pulled out from the hypodermic needle and is replaced by the catheter for fluid delivery when the needle reaches a target tissue, and after the catheter been placed the hypodermic needle also been drew out just as the traditional procedure.

### BRIEF DESCRIPTION OF THE DRAWINGS

FIG. 1 depicts the various structures of the spinal column, wherein a hypodermic needle punctures those tissues to deliver anesthetics into the epidural space during performance of epidural anesthesia.

FIG. 2 shows the light pathway of the syringe device.

FIG. 3 shows a spectrum data of a pork sample produced by the light analyzer.

FIG. 4 illustrates a diagram of single optical fiber circuit design of the device.

FIG. 5 illustrates another diagram of multiple optical fiber circuit design of the device.

### DETAIL DESCRIPTION OF THE INVENTION

The idea of this invention is derived from the concept that different tissues due to their different composition have different optical properties, such as absorbance and reflection, so we can discriminate the tissue types where the needle reaches by their distinctive optical properties. Thus we provide a new device and a new method for tissue puncture, especially for epidural anesthesia, that can position a hypodermic needle in real time through optical technology to improve the accuracy of surgery.

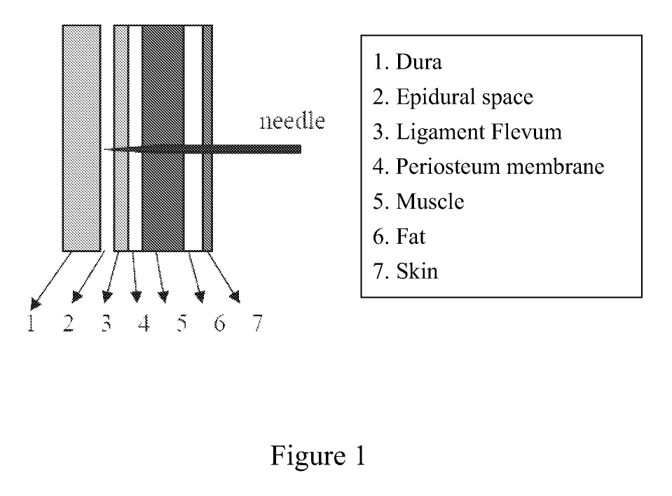

FIG. 1 shows a longitudinal profile of human posterior part and the tissue layers from outside to inside are skin, fat, muscle, periosteum membrane, ligament flavum, epidura space, and dura, but the spin is not indicated in this figure. During epidural anesthesia, the periosteum membrane is not necessary punctured.

In the present invention, the hypodermic needle used in the device is a normal clinical needle, which has a hollow inner bore to place an inner needle filled with optical fibers. The distal end of the fibers stick to the tip of inner needle, and the basal end of the fibers connect to the light source and the light analyzer.

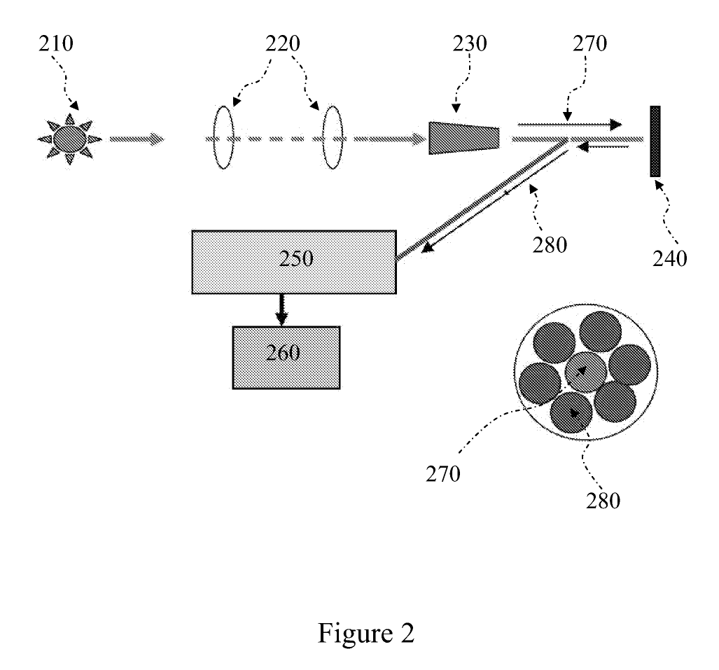

FIG. 2 shows the design of the light pathway of this syringe device. The lights emit from light source 210 transmit through lens 220 into optical fiber, and then focus on the tissue 240 by object lens 230 on the needle tip. The reflective light either passes through the same optical fiber 270 or transmits into another optical fiber 280 then to a light analyzer 250. The signal collected and analyzed by the analyzer 250 then passed to a computer 260 to be further analyzed to provides the user with the tissue type information.

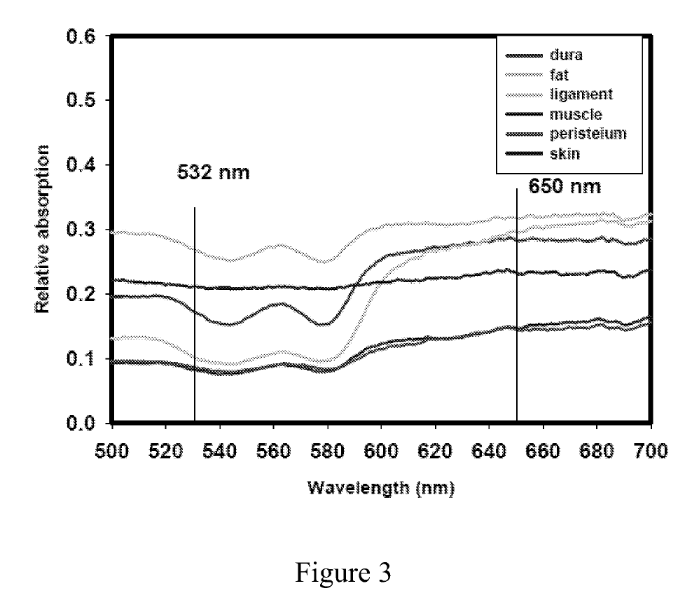

In the pilot study, a posterior pork is used for this experiment. As the FIG. 2 shows, the incident lights transmit through central optical fiber 270 and the reflective lights transmit through peripheral fibers 280. After adjustment by the analyzer a spectrum data shown in FIG. 3 is produced. When comparing the absorbency of the 650 nm and 532 nm incident lights in different tissues, the ratio of 650 nm one (Absorbency 650, Abs₆₅₀) divided by 532 nm one (Absorbency 532, Abs₅₃₂)is large than 3 in ligament flavum, however in other tissues the ratio is less than 2. Thus the absorbency ratio of these two wavelengths can be a good tool for discriminating ligament flavum from other. Also, when the needle penetrates ligament flavum into epidural space, the absorbency ratio (Abs₆₅₀/Abs₅₃₂) will droopily decrease (<2), this is a sigh that tells us to stop pushing the needle. Then we pull out the inner needle, insert a catheter and start delivering drug into that epidural space.

Actually, the absorbency ratio of Abs₆₅₀/Abs₅₈₀ is much higher than Abs₆₅₀/Abs₅₃₂ one and more suitable for tissue discrimination, but duo to there are already some commercial 532 laser light sources existing, so the Abs₆₅₀/Abs₅₃₂ one is much more convenient.

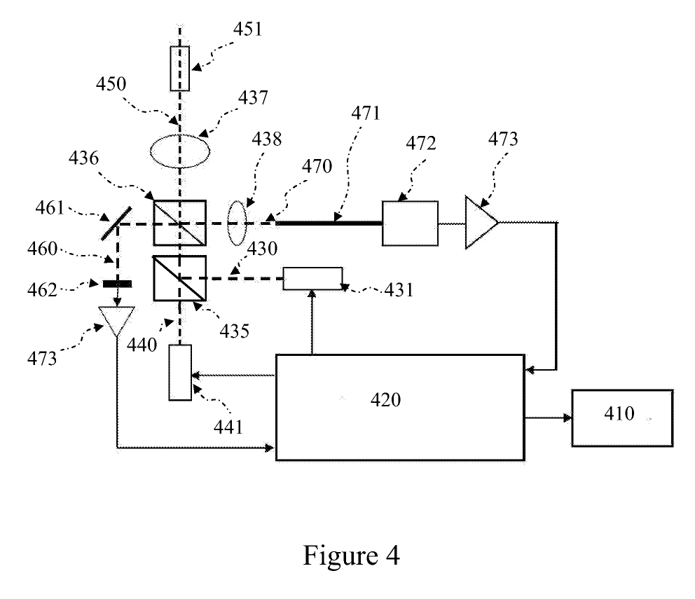

FIG. 4 illustrates a diagram of single optical fiber circuit design of the device. The control circuit 420 controls the two light sources with different wavelengths 431 and 441, wherein only one of these two light sources is on and the other is off. The two incident lights enter light paths 430 and 440 separately then split into two lights by beam splitters 435 and 436. In detail, the light from light source 431 passes through light path 430 and reflects on beam splitter 435 then split by another beam splitter 436 into two lights, one goes through light path 450 straightly and is focused by lens 437 then transmitted into optical fiber 451; the other light is reflected into light path 460 by a mirror 461 and is filtered and amplified by a photodiode 462 and a signal amplifier 473 to be an intensity criterion of the incident light. The other light source 441 is undergoes the same procedure as said above.

When the lights reach the tissues they are reflected from those tissues, and those reflective lights transmit through the same incident optical fiber 451, reflected by the beam splitter 436 and then amplified by a photomultiplier tube 472 and another signal amplifier 473.

The incident light signals and the reflective light signals pass to the control circuit 420 to be further processed, such as Abs₆₅₀/Abs₅₃₂ ratio analysis, and the result will be displayed on a signal displayer 410, ex. a monitor. Also, the user can define a notification threshold about the Abs₆₅₀/Abs₅₃₂ ratio to inform the user whether the hypodermic needle reaches the target tissue or not. The optical fiber design in FIG. 4 is different from FIG. 2, wherein FIG. 2 shows a hypodermic needle filled with multiple fibers. In practical, the needle used in epidural anesthesia is so narrow that it can only be filled with only one fiber, thus we use either a single plastic fiber or a multimode fiber glass fiber to transmit the incident lights and reflective lights simultaneity. Also these optical components will be coated with some anti-reflection matters to improve the light transmission accuracy.

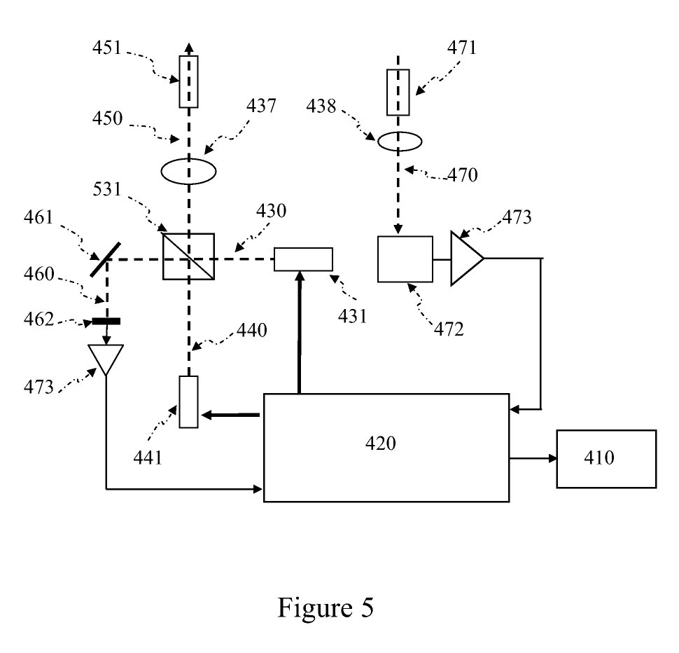

FIG. 5 illustrates another diagram of multiple optical fiber circuit design of the device, wherein some fibers 451 are used for incident light transmission and the other fibers 471 are used for reflective light receiving. The working principle of this design is as same as that in FIG. 4.

The present invention also provides a method that can position a hypodermic needle in real time through optical technology. This method adopts the same idea of the above mention, which uses the ratio between two absorbencies at different wavelength to discriminate the tissues, and achieves the goal by using said syringe device, especially in epidural anesthesia.

One skilled in the art readily appreciates that the present invention is well adapted to carry out the objects and obtain the ends and advantages mentioned, as well as those inherent therein. The tissue type discriminable syringe device and the method for positioning a hypodermic needle in real time through optical technology are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A tissue type discriminable syringe device comprising: (a) a hypodermic needle having an outer sheath that has a sharp distal tip for puncturing tissue, wherein said outer sheath defines a hollow inner bore; (b) an inner removable optical fiber within said hollow inner bore such that the light can be transmitted through the fiber into the needle and onto the contacted tissue to stimulate a reflective light by the tissue, and also the reflective light can be transmitted back through the needle and the optical fiber; (c) an catheter suitable for insertion into the needle to replace the optical fiber and for fluid delivery; (d) a light source coupled to the optical fiber for production incident light that transmitted in the fiber; and (e) a light analyzer coupled to the optical fiber for receiving and detecting the reflective light emission from the tissue.

2. The device of claim 1 wherein the optical fiber is pulled out from the hypodermic needle and is replaced by the catheter when the needle reaches a target tissue.

3. The device of claim 1 wherein the hypodermic needle and the catheter are suitable for clinical use.

4. The device of claim 1 wherein the optical fiber is at least one strip.

5. The device of claim 1 wherein the optical fiber is a single mode fiber, multi mode fibers, or branch fiber.

6. The device of claim 1 wherein the light source provides at least two different wavelengths.

7. The device of claim 6 wherein the light source is laser.

8. The device of claim 1 wherein the light analyzer analyses the ratio of one reflective light to another, wherein the two reflective lights are produced by two incident lights on different wavelengths targeting to the same tissue, to provide the tissue type information.

9. The device of claim 8 wherein one wavelength ranges from 620 nm to 680 nm and the other ranges from 520 nm to 600 nm.

10. The device of claim 9 wherein one wavelength is 650 nm and the other is 532 nm or 580 nm.

11. The device of claim 1 which is applied on epidural anesthesia.

12. A method to position a hypodermic needle through optical technology comprising: (a) inserting a removable optical fiber into a hollow hypodermic needle which has a sharp distal tip for puncturing tissue; (b) connecting the optical fiber with a light source and a light analyzer, wherein the optical fiber is capable of transmitting the incident light to the tissue and collecting the reflective light to the light analyzer; (c) transmitting at least two different incident lights to the tissue; and (d) using the light analyzer to analyze the light absorbency of the tissue.

13. The method of claim 12 wherein the light analyzer analyses the ratio of one reflective light to another, wherein the two reflective light are produced by two incident lights on different wavelengths from the light source targeting to the same tissue, to provide the tissue type information.

14. The method of claim 12 wherein the optical fiber is pulled out from the hypodermic needle and is replaced by a catheter for fluid delivery when the needle reaches a target tissue.

15. The method of claim 12 which is applied on epidural anesthesia.
